(19) United States
(12) Patent Application Publication
(10) Pub. No.: US 2026/0159848 A1
(43) Pub. Date: Jun. 11, 2026

(54) METHOD FOR ENHANCING PLANT TRANSFORMATION EFFICIENCY BY USING AGROBACTERIUM CARRYING virG MUTANT GENE AND NahG GENE

(71) Applicant: NULLA BIO INC., Gyeongsangnam-do (KR)

(72) Inventors: JIN-HEE JEONG, Gyeongsangnam-do (KR); EUN-YOUNG JEON, Gyeongsangnam-do (KR)

(21) Appl. No.: 19/307,374

(22) Filed: Aug. 22, 2025

Related U.S. Application Data
(63) Continuation-in-part of application No. PCT/KR2025/009802, filed on Jul. 8, 2025.

(30) Foreign Application Priority Data
Dec. 10, 2024 (KR) 10-2024-0182195

(51) Int. Cl. C12N 15/82; A01H 4/00; C07K 14/195; C12N 9/02; C12N 9/10; C12N 9/78

(52) U.S. Cl. CPC C12N 15/8205; A01H 4/008; C07K 14/195; C12N 9/0073; C12N 9/1096; C12N 9/78; C12Y 114/13001; C12Y 206/01019; C12Y 305/99007

(57) ABSTRACT

A method for enhancing transformation efficiency of a plant includes transforming a plant cell with a bacteria of the genus Agrobacterium carrying virG^{N54D} gene and NahG (salicylate hydroxylase) gene. The method is based on the discovery that introducing both a constitutively active virG mutant gene (virG^{N54D}) and a salicylic acid-degrading gene (NahG) into an Agrobacterium strain synergistically increases its ability to deliver a target gene into a plant cell. The provided strains can be used to improve transformation efficiency in a wide variety of plants, including recalcitrant species, and are useful for the production of transgenic plants and for applications in plant genome editing.

### CROSS-REFERENCE TO RELATED APPLICATION AND CLAIM OF PRIORITY

The present application is a continuation in part of application to International Application No. PCT/KR2025/009802 with an International Filing Date of Jul. 8, 2025, which claims the benefit of Korean Patent Application No. 10-2024-0182195 filed on Dec. 10, 2024 at the Korean Intellectual Property Office, the disclosures of which are incorporated by reference herein in their entirety.

### REFERENCE TO AN ELECTRONIC SEQUENCE LISTING

A sequence listing electronically submitted on Aug. 22, 2025 as a XML file named 20250822_S59225GR07_TU_SEQ.XML, created on Aug. 6, 2025 and having a size of 10,804 bytes, is incorporated herein by reference in its entirety.

### BACKGROUND

### 1. Technical Field

The present invention relates to a method for enhancing the transformation efficiency of a plant by using Agrobacterium carrying virG^{N54D}, which is the virG mutant gene, and the NahG (salicylate hydroxylase) gene.

This study was carried out with the support of the Startup Growth Technology Development Program of the Korea Technology and Information Promotion Agency for SMEs, under the Ministry of SMEs and Startups of South Korea (Project No. RS-2024-00468098).

### 2. Background Art

Agrobacterium is a natural genetic tool usefully employed in the field of plant biotechnology to introduce a specific foreign gene into a plant. Initially, it was reported that Agrobacterium contains a Ti (tumor-inducing) plasmid, which delivers and expresses oncogenes in plant tissues, thereby causing crown gall disease. However, as plant biotechnology has advanced, it has been discovered that the Ti plasmid can be separated into a helper plasmid that does not contain oncogenes and a binary vector. This has led to the development of technologies that safely introduce a specific gene into a plant. Nevertheless, Agrobacterium-mediated transformation is still not effectively achieved in many plant species.

In addition, Agrobacterium is influenced by various plant-derived substances, receiving both positive and negative effects. For example, certain compounds from plants can promote its specific innate abilities, while others can inhibit its growth. Plant-derived substances known to exhibit negative effects on Agrobacterium include GABA (γ-aminobutyric acid), ethylene, IAA (indole acetic acid), and salicylic acid. Accordingly, a need exists for improved Agrobacterium-mediated transformation methods that can overcome the inhibitory effects of plant-derived compounds like salicylic acid to increase the efficiency of foreign gene delivery.

Salicylic acid, in particular, is a plant hormone that activates plant defense responses against various pathogens. It is known to inhibit the replication of the Ti plasmid in Agrobacterium by suppressing quorum sensing signals. In addition, it directly inhibits the VirA/VirG two-component signaling system, thereby suppressing the expression of Agrobacterium's vir gene. Salicylic acid can be oxidized and broken down into catechol by salicylate hydroxylase or salicylate 1-monooxygenase. Therefore, the inventors of the present invention sought to investigate whether the activity of salicylic acid in plants could be altered, or whether the efficiency of foreign gene delivery could change, when Agrobacterium carrying salicylic acid-degrading enzyme is used to induce plant transformation.

Meanwhile, Korean Patent Registration No. 0948980 discloses “Method for increasing transformation efficiency of garlic using Agrobacterium and transgenic garlic produced by the method,” and Korean Patent Registration No. 0927135 discloses “Method for improving plant transformation efficiency using highly concentrated Agrobacterium and vacuum infiltration.” However, prior to the present invention, the synergistic effect of combining a constitutively active virG mutant, such as virG^{N54D}, with the salicylic acid-degrading NahG gene in an Agrobacterium strain to enhance plant transformation efficiency had not been disclosed.

### SUMMARY

The present invention is devised in view of the above-mentioned needs. In the present invention, a vector expressing the virG^{N54D} gene and t]NahG (salicylate hydroxylase) gene is constructed and introduced into Agrobacterium. Then, an Agrobacterium strain is further prepared by introducing a vector expressing the target gene (GFP or RUBY). Using this Agrobacterium, plants (tobacco or hemp) are transformed, and the transformation efficiency for the target gene was analyzed. As a result, it was found that the transformation efficiency using Agrobacterium carrying the virG^{N54D} gene and NahG gene is higher than the efficiency of the control group using Agrobacterium without the NahG gene, and the present invention is completed accordingly.

To solve the problems that are described above, the present invention provides a method for enhancing the transformation efficiency of a plant, comprising transforming a plant cell with an Agrobacterium strain carrying the virG^{N54D} gene and the NahG (salicylate hydroxylase) gene.

The present invention further provides a composition for enhancing transformation efficiency of a plant, comprising as an active ingredient bacteria of the genus Agrobacterium carrying the virG^{N54D} gene and NahG gene.

The present invention further provides bacteria of the genus Agrobacterium carrying the virG^{N54D} gene and NahG gene, which are used to enhance plant transformation efficiency.

The present invention still further provides a method for producing a plant with enhanced transformation efficiency, comprising: transforming a plant cell with bacteria of the genus Agrobacterium carrying the virG^{N54D} gene and NahG gene; and regenerating a transgenic plant from the transformed plant cell.

The use of the Agrobacterium strain of the present invention, carrying the virG^{N54D} gene and the NahG (salicylate hydroxylase) gene, allows for the effective and stable delivery of a gene of interest into a plant, thereby enhancing transformation efficiency. Accordingly, the present invention is highly applicable to the production of a transgenic plant and to the field of plant genome editing.

### BRIEF DESCRIPTION OF THE DRAWINGS

FIG. 1 is a schematic diagram of the vectors used in the present invention, showing the GabT and AcdS gene expression vector (Tv-GE), the GabT, AcdS, and virG^{N54D} gene expression vector (Tv-GEV), the NahG gene expression vector (Tv-S), the GabT, AcdS, and NahG gene expression vector (Tv-GES), the virG^{N54D} and NahG gene expression vector (Tv-VS), and the GabT, AcdS, virG^{N54D}, and NahG gene expression vector (Tv-GEVS).

FIGS. 2A to 2C show the results of measuring (FIG. 2A) salicylic acid (SA) degradation activity, (FIG. 2B) GABA aminotransferase activity, and (FIG. 2C) ACC deaminase activity of Agrobacterium strain EHA105 each harboring the vectors of the present invention (Tv-GE, Tv-GEV, Tv-S, Tv-GES, Tv-VS, or Tv-GEVS). GV3101 (pMP90) refers to Agrobacterium strain GV3101 harboring the pMP90 vector, and EHA105 (EV) refers to Agrobacterium strain EHA105 harboring an empty vector.

FIGS. 3A and 3B show the results of evaluating the transformation efficiency for the GFP (green fluorescent protein) gene in tobacco plants using Agrobacterium strain EHA105, harboring one of the vectors of the present invention (Tv-GE, Tv-GEV, Tv-S, Tv-GES, Tv-VS, or Tv-GEVS). Panel A is a Western blot result showing the expression level of GFP protein in tobacco leaves, and Panel B shows the quantification of the Western blot results. LBA4404: wild-type Agrobacterium strain LBA4404; EHA105 (pRiA4-VIR): Agrobacterium strain EHA105 harboring the VIR gene expression vector; LBA4404 (pRiA4-VIR): Agrobacterium strain LBA4404 harboring the VIR gene expression vector.

FIGS. 4A to 4C show the results of evaluating the transformation efficiency for the RUBY gene in hemp plants using Agrobacterium strain EHA105, harboring one of the vectors of the present invention (Tv-GE, Tv-GEV, Tv-S, Tv-GES, Tv-VS, or Tv-GEVS). Panel A is a photograph showing betalain accumulation in immature hemp embryos, and Panels B and C show the results of measuring betalain content through a colorimetric assay. Mock: negative control.

### DETAILED DESCRIPTION

Unless defined otherwise, all technical and scientific terms used herein have the same meaning as commonly understood by one of ordinary skill in the art to which this invention belongs.

The term “enhancing transformation efficiency” as used herein refers to a statistically significant increase in the transfer or expression of a target gene in plant cells or tissues, as compared to a control Agrobacterium strain that does not carry both the virG^{N54D} gene and the NahG gene. The efficiency can be measured, for example, by the level of reporter protein expression or the frequency of stable transformation events.

The term “Agrobacterium strain” is used to refer to bacteria of the genus Agrobacterium.

The term “target gene” as used herein refers to any nucleic acid sequence of interest intended for introduction into a plant cell, including but not limited to, genes encoding proteins, non-coding RNAs, and regulatory sequences.

To achieve object of the present invention, the present invention provides a method for enhancing transformation efficiency of a plant, comprising transforming a plant cell with bacteria of the genus Agrobacterium carrying the virG^{N54D} gene and NahG (salicylate hydroxylase) gene.

In the present invention, the term “transformation efficiency” means the ability to stably and effectively deliver a desired target gene into a plant.

In the method for enhancing transformation efficiency of a plant according to the present invention, the NahG gene may be the NahG gene derived from Pseudomonas putida, consisting of the nucleotide sequence of SEQ ID NO: 1, but is not limited thereto.

In addition, the virG^{N54D} gene is a mutant gene of the virG gene of Agrobacterium tumefaciens, in which the 54th amino acid, asparagine (N), is substituted with aspartic acid (D). It may consist of the nucleotide sequence of SEQ ID NO: 2, but is not limited thereto.

Furthermore, the genus Agrobacterium is preferably Agrobacterium tumefaciens, and more preferably the Agrobacterium tumefaciens strain EHA105, but is not limited thereto.

In the method according to the present invention, the “plant cell” to be transformed using the Agrobacterium strain carrying the virG^{N54D} gene and the NahG gene can be derived from a wide variety of plant species. The plant cell may be a cultured cell, a cultured tissue, a cultured organ, or a whole plant. “Plant tissue” refers to differentiated or undifferentiated tissues of plants, including, but not limited to, cotyledons, embryos, hypocotyls, roots, stems, leaves, pollen, seeds, female tissues, and various forms of cells used in culture such as single cells, protoplasts, immature embryos, shoots, and callus tissues. Plant tissue may be in planta or in organ culture, tissue culture, or cell culture conditions.

In one embodiment of the method according to the present invention, the plant may be a dicotyledonous plant such as tobacco, hemp, tomato, Arabidopsis, potato, eggplant, pepper, burdock, crown daisy, lettuce, balloon flower, spinach, Swiss chard, sweet potato, celery, carrot, water parsley, parsley, Chinese cabbage, cabbage, mustard greens, watermelon, melon, cucumber, pumpkin, gourd, strawberry, soybean, mung bean, kidney bean, or pea; or a monocotyledonous plant such as corn, barley, wheat, rice, oat, rye, or sugarcane. Preferably, the plant is dicotyledonous, and more preferably tobacco (Nicotiana benthamiana) or hemp (Cannabis sativa), but is not limited thereto.

In addition, the method of the present invention includes regenerating a transgenic plant from the transformed plant cell. Any method known in the art may be used for regenerating a transgenic plant from the transformed plant cell.

Specifically, the method for enhancing transformation efficiency of a plant according to one embodiment of the present invention may include the following steps of:
(a) germinating tobacco seeds and culturing them for 25 to 35 days;
(b) obtaining fully expanded leaves from the cultured tobacco plant;
(c) introducing a target gene expression vector into Agrobacterium tumefaciens strain carrying the virG^{N54D} gene and NahG gene, followed by culturing to prepare a culture solution; and
(d) infiltrating the culture solution from step (c) into the abaxial side of the plant leaves obtained in step (b), followed by co-cultivation for 94 to 98 hours to have transformation.

Alternatively, the method may include the following steps of:
(a) germinating hemp seeds and culturing them for 25 to 35 days;
(b) obtaining an immature embryo from the cultured hemp plant;
(c) introducing a target gene expression vector into Agrobacterium tumefaciens strain carrying the virG^{N54D} gene and NahG gene, followed by culturing to prepare a culture solution; and
(d) immersing the immature embryo obtained in step (b) in the culture solution from step (c) for 30 to 40 minutes, followed by co-cultivation for 70 to 74 hours to have transformation. However, the method is not limited to those steps.

The target gene refers to a portion of DNA intended to be delivered through the present invention and is not limited to any specific type of gene. It may include both coding and non-coding regions. Those skilled in the art can select the target gene according to the purpose and the transgenic plant to be produced.

The present invention further provides a composition for enhancing transformation efficiency of a plant, comprising as an active ingredient bacteria of the genus Agrobacterium carrying the virG^{N54D} gene and NahG gene.

In the composition for enhancing transformation efficiency of a plant according to the present invention, the virG^{N54D} gene, NahG gene, and bacteria of the genus Agrobacterium are as described above.

In the composition for enhancing transformation efficiency of a plant according to the present invention, the bacteria of the genus Agrobacterium may be further carrying the GABA aminotransferase gene (GabT) and ACC deaminase gene (AcdS), but are not limited thereto.

The present invention further provides bacteria of the genus Agrobacterium carrying the virG^{N54D} gene and NahG gene, which are used to enhance plant transformation efficiency.

As for the bacteria of the genus Agrobacterium according to the present invention, the virG^{N54D} gene, NahG gene, and bacteria of the genus Agrobacterium are as described above.

In one embodiment of the bacteria of the genus Agrobacterium according to the present invention, the bacteria of the genus Agrobacterium may be further carrying the GABA aminotransferase gene (GabT) and ACC deaminase gene (AcdS), but are not limited thereto.

The present invention still further provides a method for producing a plant with enhanced transformation efficiency, comprising:
transforming a plant cell with bacteria of the genus Agrobacterium carrying the virG^{N54D} gene and NahG gene; and
regenerating a transgenic plant from the transformed plant cell.

In the method for producing a plant with enhanced transformation efficiency according to the present invention, the virG^{N54D} gene, NahG gene, and bacteria of the genus Agrobacterium are as described above.

Hereinafter, the present invention will be described in detail with reference to examples. However, the following examples are provided only to illustrate the present invention and are not intended to limit the scope of the invention to those examples.

While the examples herein utilize the pBBR1-MCS5B broad-host-range vector, it is to be understood that the invention is not so limited. One skilled in the art will recognize that other suitable vectors can be used to carry the virG^{N54D} and NahG gene expression cassettes. Such vectors include, but are not limited to, other broad-host-range plasmids compatible with Agrobacterium, such as those from the pRK2 or pVS1 replicon groups.

Furthermore, while the efficacy of the invention has been demonstrated in dicotyledonous plants such as tobacco, hemp, and tomato, the method is contemplated to be effective across a wide range of plant species, including other recalcitrant dicots and various monocots. The ability to overcome plant defense mechanisms mediated by salicylic acid is a general mechanism that is not limited to a specific plant species.

### EXAMPLES

### Example 1. Preparation of Vector Expressing virG^{N54D} and NahG Gene

In the present invention, the nucleotide sequence of the salicylate hydroxylase (NahG) gene (SEQ ID NO: 1) was determined from the GenBank database (Accession No. M60055.1), and the nucleotide sequence of the virG^{N54D} gene (SEQ ID NO: 2) was amplified from the Addgene vector (#123187) and used in the experiments. Furthermore, the nucleotide sequence of the GABA aminotransferase (GabT) gene (SEQ ID NO: 3) was determined from the GenBank database (Accession No. 948067), and the nucleotide sequence of the ACC deaminase (AcdS) gene (SEQ ID NO: 4) was determined from the GenBank database (Accession No. AY823987) and used in the experiments.

The NahG gene expression cassette, synthesized via a gene synthesis service (Genewiz), was inserted into the cloning site of the broad-host-range vector pBBR1-MCS5B using Golden Gate cloning to prepare the NahG gene expression vector (Tv-S).

Additionally, the GabT and AcdS gene co-expression cassette, synthesized via a gene synthesis service (GenScript), was inserted into the cloning site of pBBR1-MCS5B using Golden Gate cloning to prepare the GabT and AcdS gene expression vector (Tv-GE).

Additionally, using virG-F2 primer (5′-cagtcGGTCTCatagctgtaacctcgaagcgt-3′: SEQ ID NO: 5) and virG-R1 primer (5′-cagtcGGTCTCaAAGCccgtcttggtggtcagtgtg-3′: SEQ ID NO: 6), the virG^{N54D} gene expression cassette was prepared and inserted together with the GabT and AcdS gene co-expression cassette into the cloning site of pBBR1-MCS5B via Golden Gate cloning to generate the GabT, AcdS, and virG^{N54D} gene expression vector (Tv-GEV).

Additionally, the GabT and AcdS gene co-expression cassette and the NahG gene expression cassette were inserted into the cloning site of pBBR1-MCS5B via Golden Gate cloning to prepare the GabT, AcdS, and NahG gene expression vector (Tv-GES).

Additionally, the virG^{N54D} gene expression cassette and the NahG gene expression cassette were inserted into the cloning site of pBBR1-MCS5B via Golden Gate cloning to prepare the virG^{N54D} and NahG gene expression vector (Tv-VS).

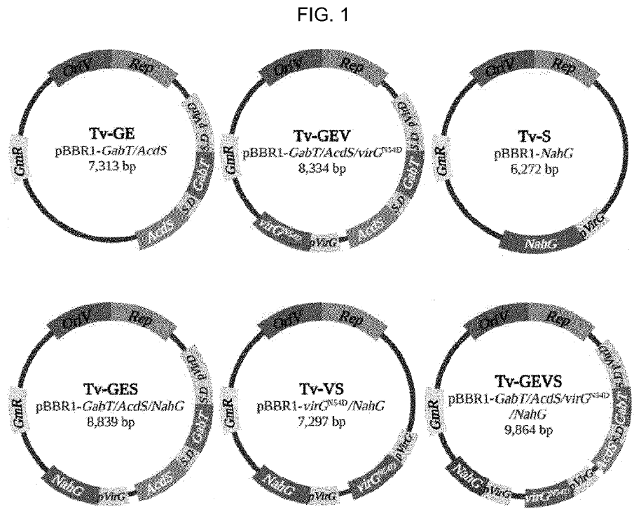

Additionally, the GabT and AcdS gene co-expression cassette, the virG^{N54D} gene expression cassette, and the NahG gene expression cassette were inserted into the cloning site of pBBR1-MCS5B via Golden Gate cloning to prepare the GabT, AcdS, virG^{N54D}, and NahG gene expression vector (Tv-GEVS) (FIG. 1, Table 1).

TABLE 1
Vectors used in the present invention

| Vector | Description |
|---|---|
| Tv-GE | Inserted with GabT, AcdS genes |
| Tv-GEV | Inserted with GabT, AcdS, virG^{N54D} genes |
| Tv-S | Inserted with NahG gene |
| Tv-GES | Inserted with GabT, AcdS, NahG genes |
| Tv-VS | Inserted with virG^{N54D}, NahG genes |
| Tv-GEVS | Inserted with GabT, AcdS, virG^{N54D}, NahG genes |

Each of the vectors in Table 1 was introduced into Agrobacterium tumefaciens EHA105 strain by electroporation, and then cultured in LB (Luria-Bertani) medium containing 50 mg/L gentamycin and 50 mg/L rifampicin.

### Example 2. Measurement of Salicylic Acid (SA) Degradation Activity

The salicylate hydroxylase gene (NahG) encodes an enzyme that oxidizes salicylic acid and breaks it down into catechol. Accordingly, the salicylic acid degradation activity was measured by assessing the catechol content in Agrobacterium EHA105 strain into which each of the following vectors was introduced: GabT and AcdS gene expression vector (Tv-GE), GabT, AcdS, and virG^{N54D} gene expression vector (Tv-GEV), NahG gene expression vector (Tv-S), GabT, AcdS, and NahG gene expression vector (Tv-GES), virG^{N54D} and NahG gene expression vector (Tv-VS), and GabT, AcdS, virG^{N54D}, and NahG gene expression vector (Tv-GEVS).

Specifically, each Agrobacterium strain was cultured until OD600 reached 0.8, then BugBuster Master Mix (Novagen) and EDTA-free Protease Inhibitor Cocktail (Roche) were added for bacterial protein extraction and subjected to lysis at room temperature for 20 minutes. The samples were then centrifuged at 10,000 rpm for 20 minutes, and the supernatant was transferred to a new tube. Protein concentration in the supernatant was measured using the BCA protein assay (Thermo Scientific). Next, to the reaction mixture (containing 200 μM salicylic acid, 200 μM β-Nicotinamide adenine dinucleotide hydrate (NADH), 20 μM flavin adenine dinucleotide disodium salt hydrate (FAD), and 33 mM potassium phosphate buffer, pH 7.0), the supernatant of each culture solution, whose concentration was measured above, was added in an amount of 50 μg based on the protein concentration, and the salicylic acid degradation reaction was induced at 25° C. After the reaction, the remaining salicylic acid formed a purple salicylic acid-FeCl₃ complex with ferric chloride (FeCl₃). The absorbance of this purple solution was measured at wavelength of 560 nm to quantify the amount of salicylic acid remaining in the reaction mixture, thereby calculating the salicylic acid degradation activity. As a control, Agrobacterium tumefaciens GV3101 strain harboring the pMP90 vector and Agrobacterium tumefaciens EHA105 strain harboring the pBBR1 vector were used.

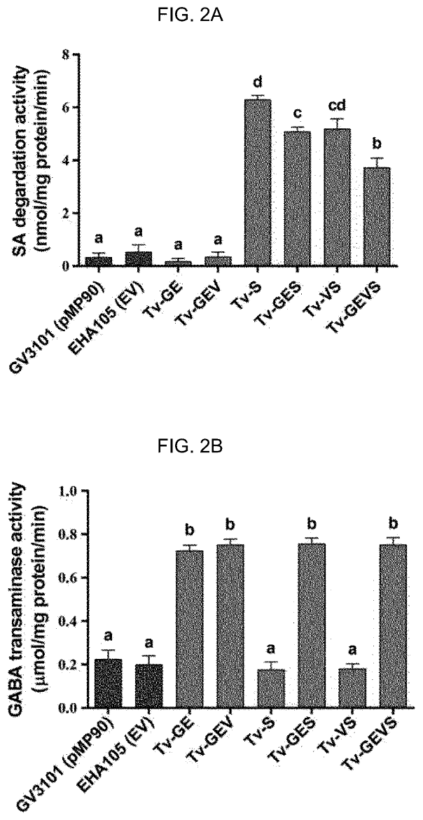

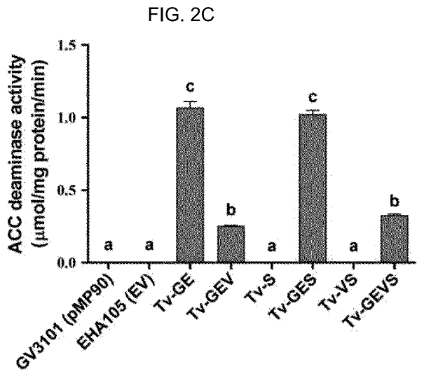

As a result, it was found that the salicylic acid degradation activity of Agrobacterium EHA105 strains harboring the NahG gene expression vector (Tv-S), GabT, AcdS, NahG gene expression vector (Tv-GES), virG^{N54D} and NahG gene expression vector (Tv-VS), or GabT, AcdS, virG^{N54D}, NahG gene expression vector (Tv-GEVS) was significantly increased compared to the control strains. This indicated that the introduced NahG gene functions properly within the Agrobacterium (FIG. 2A).

### Example 3. Measurement of GABA Aminotransferase Activity

The GABA aminotransferase (GabT) gene encodes an enzyme that degrades GABA into glutamate. Accordingly, the GABA aminotransferase activity was measured by assessing glutamate content in Agrobacterium EHA105 strains, each harboring one of the following vectors: GabT and AcdS gene expression vector (Tv-GE), GabT, AcdS, virG^{N54D} gene expression vector (Tv-GEV), NahG gene expression vector (Tv-S), GabT, AcdS, NahG gene expression vector (Tv-GES), virG^{N54D} and NahG gene expression vector (Tv-VS), and GabT, AcdS, virG^{N54D}, NahG gene expression vector (Tv-GEVS).

Specifically, each Agrobacterium strain was cultured to an OD600 of 0.8, then lysed at room temperature for 20 minutes using the bacterial protein extraction reagent BugBuster Master Mix (Novagen) along with EDTA-free Protease Inhibitor Cocktail (Roche). The lysate was centrifuged at 10,000 rpm for 20 minutes, and the supernatant was transferred to a new tube. Protein concentration in the supernatant was measured using the BCA protein assay kit (Thermo Scientific). Next, to the reaction mixture (0.1 M NaOH, 1 mM pyridoxal phosphate, 10 mM 2-ketoglutarate, 10 mM GABA), the supernatant of each culture solution, whose concentration was measured above, was added in an amount of 100 μg based on the protein concentration, and the GABA degradation reaction was induced by incubating the mixture at 37° C. for 10 minutes. Afterwards, the glutamate content was measured using a glutamate assay kit (Sigma). Agrobacterium tumefaciens GV3101 strain harboring the pMP90 vector and Agrobacterium tumefaciens EHA105 strain harboring the pBBR1 vector were used as a control.

As a result, it was found that the GABA aminotransferase activity of Agrobacterium EHA105 strains harboring the GabT and AcdS gene expression vector (Tv-GE), GabT, AcdS, virG^{N54D} gene expression vector (Tv-GEV), GabT, AcdS, NahG gene expression vector (Tv-GES), or GabT, AcdS, virG^{N54D}, NahG gene expression vector (Tv-GEVS) was significantly increased compared to the control strains. This indicated that the introduced GabT gene functions properly within the Agrobacterium (FIG. 2B).

### Example 4. Measurement of ACC Deaminase Activity

The ACC deaminase gene (AcdS) encodes an enzyme that breaks down ACC into α-ketobutyrate and ammonia. Accordingly, the ACC deaminase activity was measured by quantifying the α-ketobutyrate content in Agrobacterium EHA105 strains, each transformed with one of the following: GabT and AcdS gene expression vector (Tv-GE), GabT, AcdS, virG^{N54D} gene expression vector (Tv-GEV), NahG gene expression vector (Tv-S), GabT, AcdS, NahG gene expression vector (Tv-GES), virG^{N54D} and NahG gene expression vector (Tv-VS), or GabT, AcdS, virG^{N54D}, NahG gene expression vector (Tv-GEVS).

Specifically, each Agrobacterium strain was cultured to an OD600 of 0.8, then further cultured for one day in DF medium containing 3 mM ACC. Cells were lysed by treatment with toluene. Then, 200 μL of the toluene-treated cell suspension was mixed with 20 μL of 0.5 M ACC and incubated at 30° C. for 30 minutes. After centrifugation at 13,000 rpm for 5 minutes, 300 μL of 0.2% (v/v) 2,4-dinitrophenylhydrazine (2,4-DNPH) was added to the supernatant and reacted for 30 minutes. The absorbance was then measured at wavelength of 540 nm to determine the α-ketobutyrate concentration. Agrobacterium tumefaciens GV3101 strain harboring the pMP90 vector and Agrobacterium tumefaciens EHA105 strain harboring the pBBR1 vector were used as a control.

As a result, it was found that the ACC deaminase activity of Agrobacterium EHA105 strains transformed with the GabT and AcdS gene expression vector (Tv-GE), GabT, AcdS, virG^{N54D} gene expression vector (Tv-GEV), GabT, AcdS, NahG gene expression vector (Tv-GES), or GabT, AcdS, virG^{N54D}, NahG gene expression vector (Tv-GEVS) was significantly increased compared to the control strains. This indicated that the introduced AcdS gene functions properly within Agrobacterium (FIG. 2C).

### Example 5. Evaluation of Gene Transformation Efficiency in Tobacco Plant

To evaluate the gene transformation efficiency in tobacco (Nicotiana benthamiana) plant, the GFP (green fluorescent protein) gene was introduced into a tobacco plant, and the transformation efficiency of the GFP gene was assessed by measuring GFP expression.

Specifically, tobacco seeds were sown in soil and grown for one month at 23° C. under a photoperiod of 16 hours light/8 hours dark to obtain fully expanded tobacco leaves. Agrobacterium tumefaciens EHA105 strains, each transformed with one of the following: GabT and AcdS gene expression vector (Tv-GE), GabT, AcdS, and virG^{N54D} gene expression vector (Tv-GEV), NahG gene expression vector (Tv-S), GabT, AcdS, and NahG gene expression vector (Tv-GES), virG^{N54D} and NahG gene expression vector (Tv-VS), or GabT, AcdS, virG^{N54D}, and NahG gene expression vector (Tv-GEVS), were further transformed by electroporation with binary vectors carrying the GFP gene (Addgene #40259, #48015). These Agrobacterium cultures were grown in LB medium to an OD600 of 0.8. Then, 1 mL of each Agrobacterium suspension was infiltrated into an area approximately 2 cm in diameter on the abaxial side (underside) of the prepared tobacco leaves using a syringe. The infiltrated leaves were incubated at 23° C. under a 16-hour light/8-hour dark photoperiod for 96 hours. GFP fluorescence signals were detected using an Azure 600 Imaging system (Azure Biosystems). Additionally, infiltrated leaf samples were excised, rapidly frozen in liquid nitrogen, and proteins were extracted from 100 mg of the plant tissues. Western blot analysis was performed using an anti-GFP polyclonal rabbit antibody (1:10,000, Abcam ab6556) to determine GFP protein expression.

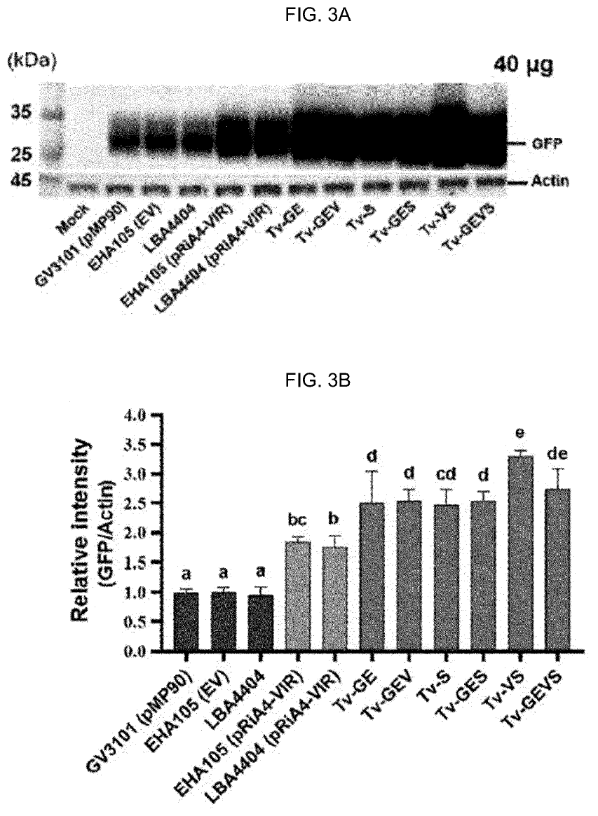

As a result, GFP expression in tobacco plant transformed with Agrobacterium EHA105 strains harboring the virG^{N54D} and NahG gene expression vector (Tv-VS) was significantly higher compared to the control or comparison groups (see FIGS. 3A and 3B).

These results indicate that the expression of virG^{N54D} and NahG genes increases the transformation efficiency of the GFP gene in tobacco plant.

### Example 6. Evaluation of Gene Transformation Efficiency in Hemp Plant

To evaluate the gene transformation efficiency in hemp (Cannabis sativa) plant, the RUBY gene, which converts tyrosine into betalain, was delivered into immature embryos of Cannabis sativa. The transformation efficiency of the RUBY gene was then assessed by measuring the betalain content.

Specifically, Cannabis sativa seeds were sown in soil and cultured at 23° C. under a 16-hour light/8-hour dark photoperiod for one month. Then, the plants were transferred to a 12-hour light/12-hour dark photoperiod to induce flowering. During this time, male and female plants were grown separately to avoid cross-contamination. When the flowers bloomed, pollen from the male flowers was manually pollinated onto the female stigmas. After 21 days, immature embryos were collected. Agrobacterium tumefaciens EHA105 strains, each harboring one of the following vectors: GabT and AcdS gene expression vector (Tv-GE), GabT, AcdS, and virG^{N54D} gene expression vector (Tv-GEV), NahG gene expression vector (Tv-S), GabT, AcdS, and NahG gene expression vector (Tv-GES), virG^{N54D} and NahG gene expression vector (Tv-VS), or GabT, AcdS, virG^{N54D}, and NahG gene expression vector (Tv-GEVS), were transformed with a binary vector containing the RUBY gene (Addgene #160908) via electroporation. The transformed Agrobacterium strains were cultured in LB medium until OD600 reached 0.8. The prepared immature Cannabis embryos were then immersed in a co-cultivation medium (half-strength (½) MS basal medium, 2% sucrose, 1% glucose, 100 μM acetosyringone, pH 5.2) containing the Agrobacterium suspension for 40 minutes. After immersion, the embryos were co-cultivated in the dark for 72 hours. Following co-cultivation, the embryos were transferred to half-strength MS basal medium supplemented with timentin and cultured for 7 days. The transformed embryos were then homogenized in liquid nitrogen to prepare powdered samples, which were used for betalain content measurement.

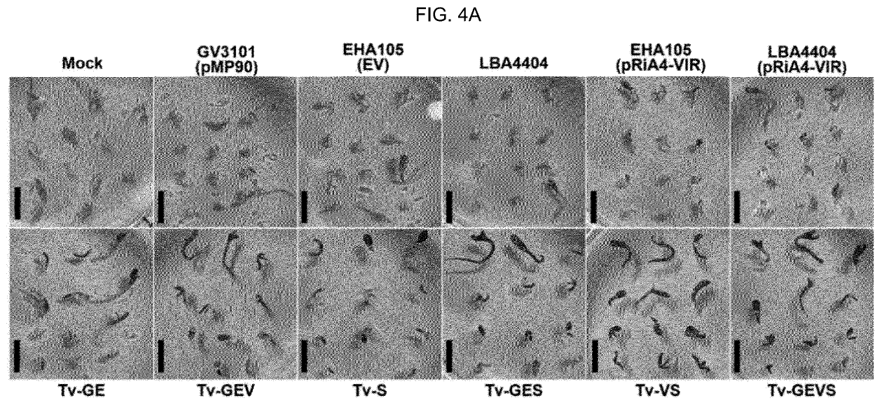

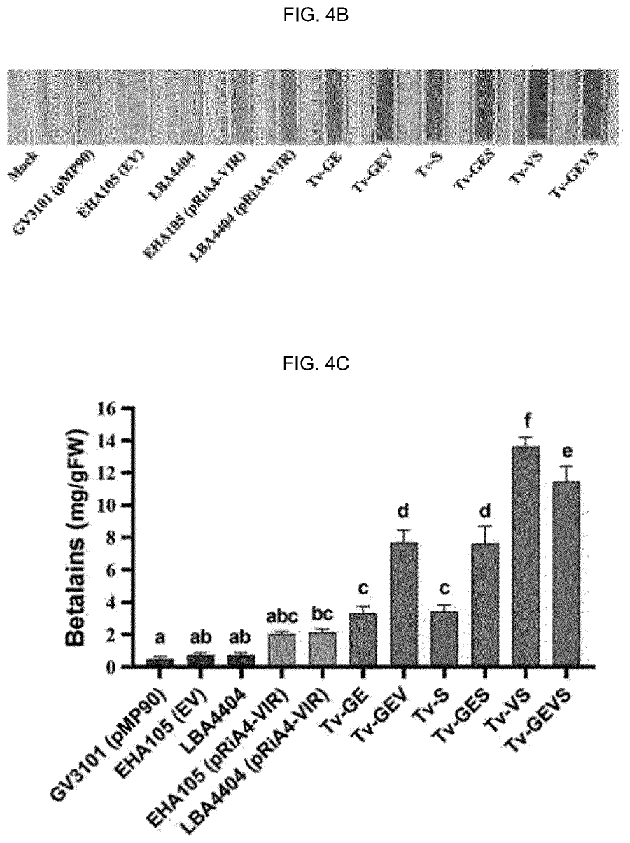

As a result, it was found that the betalain content in immature Cannabis sativa embryos transformed with Agrobacterium EHA105 strains harboring the virG^{N54D} and NahG gene expression vector (Tv-VS) was significantly increased compared to the control or comparison groups (see FIGS. 4A to 4C).

These results demonstrate that the expression of virG^{N54D} and NahG genes enhances the transformation efficiency of the RUBY gene in Cannabis sativa plants.

What is claimed is:

1. A method for enhancing transformation efficiency of a plant, the method comprising:
transforming a plant cell with a bacteria of the genus Agrobacterium carrying virG^{N54D} gene and NahG (salicylate hydroxylase) gene.

2. The method according to claim 1, wherein the NahG gene consists of the nucleotide sequence of SEQ ID NO: 1.

3. The method according to claim 1, wherein the virG^{N54D} gene consists of the nucleotide sequence of SEQ ID NO: 2.

4. The method according to claim 1, wherein the bacteria of the genus Agrobacterium are Agrobacterium tumefaciens.

5. The method according to claim 1, wherein the transforming comprises:
germinating tobacco seeds and culturing them for 25 to 35 days;
obtaining fully expanded leaves from the cultured tobacco plant;
introducing a target gene expression vector into Agrobacterium tumefaciens strain carrying virG^{N54D} gene and NahG gene;
culturing the Agrobacterium tumefaciens strain to prepare a culture solution; and
infiltrating the culture solution into an abaxial side of the fully expanded leaves, followed by co-cultivation to have transformation.

6. The method according to claim 1, wherein the transforming comprises:
germinating hemp seeds and culturing them for 25 to 35 days;
obtaining an immature embryo from the cultured hemp plant;
introducing a target gene expression vector into Agrobacterium tumefaciens strain carrying virG^{N54D} gene and NahG gene;
culturing the Agrobacterium tumefaciens strain to prepare a culture solution; and
immersing the immature embryo obtained in the culture solution, followed by co-cultivation to have transformation.

7. A composition for enhancing transformation efficiency of a plant, comprising as an active ingredient bacteria of the genus Agrobacterium carrying virG^{N54D} gene and NahG (salicylate hydroxylase) gene.

8. The composition according to claim 7, wherein the bacteria of the genus Agrobacterium are additionally carrying GABA aminotransferase gene (GabT) and ACC deaminase gene (AcdS).

9. A method for producing a plant with enhanced transformation efficiency, the method comprising:
transforming a plant cell with bacteria of the genus Agrobacterium carrying virG^{N54D} gene and NahG (salicylate hydroxylase) gene; and
regenerating a transgenic plant from the transformed plant cell.


SEQUENCE LISTING (6 sequences)

SEQ ID NO: 1 - DNA, 1305 bp, Pseudomonas putida
  source 1..1305: mol_type = genomic DNA
atgaaaaacaataaacttggcttgcgcatcggtatcgtcggcggcggaatttccggcgttgccttagcactggaactctgccgctactcccatatccaggtacagctgttcgaggctgcgccggctttcggtgaggtcggtgccggcgtgtcctttggccccaacgcggtgcgcgccattgtcggcctgggcttgggcgaggcctacctgcaggttgccgaccgtacttcggagccctgggaggacgtgtggttcgaatggcggcgcggcagcgatgccagctatctgggagccaccatcgctccgggcgtgggccagtcctcggtacaccgggcggatttcatcgacgccctagtaactcacctcccagaaggtatcgcccaattcgggaagcgcgccacccaggtcgagcagcagggtggcgaagtgcaagtgctgttcaccgacggcacagagtaccgctgcgaccttctgatcggtgccgacggaatcaagtcagcgctccgtagccatgtgctggaaggtcaggggctggccccacaagtgccgcgattcagcggcacctgtgcctatcgggggatggtcgacagcctgcatctgcgagaagcctatcgggcccatggcatcgacgagcacttggtggacgtgccgcagatgtacctagggctcgacggccatatcctcacctttccagtgaggaatggcggcatcatcaacgtggtggctttcatctccgaccgtagcgagccgaagccgacctggcctgcggatgccccttgggtgcgtgaggcgagccagcgcgagatgctcgatgccttcgcgggttggggggatgccgcgcgcgccctgctggagtgcatcccggcaccaactctctgggcactgcatgacctggcggagctgccgggctacgtgcacggtcgggtcgtcctgatcggcgacgcagctcacgccatgctgccgcaccaaggtgccggtgctggccaagggcttgaggacgcctacttcctcgcccgcctgttgggcgatacgcaggccgatgccggcaacctcgccgagctgcttgaagcctacgacgacctgcgccgccctcgtgcctgtcgcgtgcagcaaacctcctgggaaaccggcgagttatacgagttgcgcgaccccgtcgtcggtgcgaacgagcagctgctgggggaaaacctggcgacccgcttcgactggctgtggaaccacgacctcgacactgacctggccgaggcccgtgcgcggctgggttgggagcatggtggcgggggtgcgctacgtcaagggtga

SEQ ID NO: 2 - DNA, 804 bp, Agrobacterium tumefaciens
  source 1..804: mol_type = genomic DNA
atgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcggatccgtcaagccctcaactgataaaaacagcaagaggtgccggttatttctttgacgcggacgtgcaggtttcgcacggggggacgatggcagcctga

SEQ ID NO: 3 - DNA, 1281 bp, Escherichia coli
  source 1..1281: mol_type = genomic DNA
atgaacagcaataaagagttaatgcagcgccgcagtcaggcgattccccgtggcgttgggcaaattcacccgattttcgctgaccgcgcggaaaactgccgggtgtgggacgttgaaggccgtgagtatcttgatttcgcgggcgggattgcggtgctcaataccgggcacctgcatccgaaggtggtggccgcggtggaagcgcagttgaaaaaactgtcgcacacctgcttccaggtgctggcttacgagccgtatctggagctgtgcgagattatgaatcagaaggtgccgggcgatttcgccaagaaaacgctgctggttacgaccggttccgaagcggtggaaaacgcggtaaaaatcgcccgcgccgccaccaaacgtagcggcaccatcgcttttagcggcgcgtatcacgggcgcacgcattacacgctggcgctgaccggcaaggtgaatccgtactctgcgggcatggggctgatgccgggtcatgtttatcgcgcgctttatccttgcccgctgcacggcataagcgaggatgacgctatcgccagcatccaccggatcttcaaaaatgatgccgcgccggaagatatcgccgccatcgtgattgagccggttcagggcgaaggcggtttctacgcctcgtcgccagcctttatgcagcgtttacgcgctctgtgtgacgagcacgggatcatgctgattgccgatgaagtgcagagcggcgcggggcgtaccggcacgctgtttgcgatggagcagatgggcgttgcgccggatcttaccacctttgcgaaatcgatcgcgggcggcttcccgctggcgggcgtcaccgggcgcgcggaagtaatggatgccgtcgctccaggcggtctgggcggcacctatgcgggtaacccgattgcctgcgtggctgcgctggaagtgttgaaggtgtttgagcaggaaaatctgctgcaaaaagccaacgatctggggcagaagttgaaagacggattgctggcgatagccgaaaaacacccggagatcggcgacgtacgcgggctgggggcgatgatcgccattgagctgtttgaagacggcgatcacaacaagccggacgccaaactcaccgccgagatcgtggctcgcgcccgcgataaaggcctgattcttctctcctgcggcccgtattacaacgtgctgcgcatccttgtaccgctcaccattgaagacgctcagatccgtcagggtctggagatcatcagccagtgttttgatgaggcgaagcagtag

SEQ ID NO: 4 - DNA, 1017 bp, Pseudomonas putida
  source 1..1017: mol_type = genomic DNA
atgaacctgaatcgttttgaacgttatccgttgaccttcggtccatcccccatcactcccttgaaacgcctcagcgagcacctgggcggcaaggtggaactgtatgccaagcgtgaagactgcaatagcggcctggccttcggcgggaacaaaacgcgcaagctcgaatatctgattcccgaggccatcgagcaaggctgcgacaccttggtgtccatcggcggtatccagtcgaaccagacccgccaggtcgctgcggtcgccgcccacttgggtatgaagtgtgtgcttgtgcaggaaaactgggtgaactactccgacgctgtatatgaccgcgtcggcaacatcgagatgtcgcggatcatgggagcggatgtgcggcttgatgctgcaggtttcgacattggaattcggccgagctgggaaaaggccatgagcgatgtcgtggagcgcggcggcaaaccgtttccaattccggcgggctgttccgagcatccctatggagggctcgggtttgtcggcttcgctgaggaagtgcggcagcaggaaaaggagttgggcttcaagtttgactacatcgtggtctgctcggtgaccggcagtacccaggccggcatggtcgtcggtttcgcggctgacggtcgctcgaaaaacgtgatcggggtcgatgcttcggcgaaaccggagcaaaccaaggcgcagatcctgcgtatcgctcgacataccgctgaactggtggagctggggcgcgaaatcactgaagaggatgtggtgctcgatacgcgtttcgcctatccggaatatggcttgcccaacgaagggacgctggaagcgattcgcctgtgcggcagtcttgagggggtgttgaccgatccggtctacgagggcaaatccatgcacggcatgattgaaatggtacgccgcggggaattccctgacggctccaaagttctttatgcccacctgggcggcgcacctgcgttgaacgcctacagcttcttgtttcgcaacggctga

SEQ ID NO: 5 - DNA, 32 bp, synthetic construct
  source 1..32: mol_type = other DNA
cagtcggtctcatagctgtaacctcgaagcgt

SEQ ID NO: 6 - DNA, 36 bp, synthetic construct
  source 1..36: mol_type = other DNA
cagtcggtctcaaagcccgtcttggtggtcagtgtg